(19) United States
(12) Patent Application Publication
(10) Pub. No.: US 2003/0060785 A1
(43) Pub. Date: Mar. 27, 2003

(54) DISPOSABLE VAGINAL DEVICE

(76) Inventors: MICHAEL G. LAVEAN, SARANAC, MI (US); JANET M. TLAPEK, SARANAC, MI (US)

Correspondence Address: DOUGHERTY CLEMENTS & HOFER, TWO FAIRVIEW CENTER, 6230 FAIRVIEW ROAD SUITE 400, CHARLOTTE, NC, 28210, US

(21) Appl. No.: 08/671,926

(22) Filed: Jun. 27, 1996

Related U.S. Application Data
(63) Continuation of application No. 08/009,578, filed on Jan. 26, 1993, now abandoned.

(51) Int. Cl. A61F 5/44

(52) U.S. Cl. 604/330; 604/332

(57) ABSTRACT

A vaginal device such as a cervical cap diaphragm vaginal ring or other intra-vaginal device designed for delivery of biologically active materials has a first member and a second member which are selectively attached to each other, at least one of the members comprising a releasable biologically active material.

### BACKGROUND OF THE INVENTION

The present invention relates generally to a disposable vaginal device which can be used to deliver biologically active substances for a substantial period of time. More particularly, the present invention relates to a vaginal device comprising at least two parts, at least one of which can be used to deliver biologically active substances for a substantial period of time, and means for selectively assembling the parts into an operative device.

Devices which are intended to be inserted into the vagina are known for use as contraceptive barriers or as vehicles for the delivery of medicaments or spermicides. In the past, devices for the delivery of biologically active materials have included pessaries, vaginal suppositories and various devices composed of one or more reservoirs covered by a permeable surface through which the biologically active material may migrate or leave the device and then enter the body. One of the principal requirements for a device for delivering a biologically active substance to the body is that the device remain in place long enough to administer the desired dose.

Although vaginal devices have been known for some time, there is continuing development of their design and use around the world. For example, cervical caps are becoming increasingly popular for contraceptive use by the general population. Despite advances in the art, there remains a need for improved intra-vaginal devices capable of delivering a biologically active substance yet which are inexpensive and which can be self inserted. There is also a need for an improved intra-vaginal device capable of acting as a multi-purpose delivery vehicle for two or more biologically active materials. There is also a need for a vaginal device which can be readily adapted for various uses and which has a long shelf life.

The present invention represents a significant conceptual advance in the art relating to the way biologically active materials are introduced intra-vaginally. The new devices of this invention are capable of being molded into parts that may be assembled interchangeably. The present invention allows intra-vaginal, sustained delivery of biological active materials to treat a variety of sexually transmitted diseases, yeast infections, warts, ulcerations, lesions, endometriosis, hormone deficiency, etc. It will be apparent to those familiar with the art that there are a tremendous number of variations and combinations of biological active materials that could be delivered intra-vaginally in order to address a whole host of problems. For example: a preferred treatment for Chlamydia involves the administration of Tetracycline and Ampicillin for a ten day period. However, patients often fail to properly comply with the terms of medication for the ten day period. The present invention allows the delivery of Tetracycline and Ampicillin in combination in such a way that patient compliance can be removed as an issue.

In summary, the present invention provides an improved method and devices including cervical caps, diaphragms, vaginal rings and other intra-vaginal devices designed for delivery of biologically active materials. Further understanding of the present invention will be had from the following disclosure and claims taken in conjunction with accompanying drawings.

### SUMMARY OF THE INVENTION

The present invention relates to a vaginal device comprising first and second members which are selectively attached to each other at least one of said members comprising a biologically active material.

The present invention also is directed to a method of selectively attaching two or more parts of a vaginal device used for the sustained delivery of one or more biologically active materials. The device is able to sustain delivery of a wide variety of drugs simultaneously. The parts can be located in a pharmacy so that a physician would be able to prescribe the device with the appropriate “snap ins” and the pharmacist would be able to assemble it prior to dispensing the device to the user.

The parts to be used are manufactured of a biologically active loaded silicone based material that can be snap-fit together. Thus, the device is composed of two or more parts designed to be selectively snapped together prior to being inserted by the user. The parts of the current invention can be made in such a way as to promote the greatest possible amount of interchangeability.

In a preferred embodiment, the invention will be composed of a cervical cap with a plurality of grooves allowing a plurality of snap-in rings to be added prior to use. Alternative preferred embodiments include a diaphragm vaginal ring, vaginal disc or vaginal suppository, upon which there would be a plurality of snap-in parts making a whole unit prior to use. Still further preferred alternative embodiments are two or more parts that when selectively snapped together, would form a cervical cap, diaphragm, vaginal disc or a vaginal suppository. Combinations of the above alternatives can also be used.

### BRIEF DESCRIPTION OF THE DRAWINGS

FIG. 1 is a perspective view of a preferred embodiment of a cervical cap of the present invention;

FIG. 2 is a sectional view, broken away, taken along line 2-2 in FIG. 1;

FIGS. 3-5 are sectional views analogous to FIG. 2 but showing alternative preferred embodiments of rims of cervical caps of the present invention;

FIG. 6 is an exploded view in perspective, showing another alternative preferred embodiment of a cervical cap of the present invention;

FIG. 7 is a sectional view, broken away, taken along line 7-7 in FIG. 6;

FIG. 8 is an exploded view, in perspective, analogous to FIG. 6 but showing yet another alternative preferred embodiment of a cervical cap of the present invention;

FIG. 9 is a sectional view, broken away, taken along line 9-9 in FIG. 8;

FIGS. 10-11 are sectional views analogous to FIG. 9 but showing further alternative preferred embodiments of cervical caps of the present invention;

FIG. 12 is a perspective view showing a preferred embodiment of a vaginal ring of the present invention;

FIG. 13 is a sectional view, broken away, taken along line 13-13 in FIG. 12;

FIGS. 14-16 are exploded views, in perspective, of alternative preferred embodiments of vaginal suppositories of the present invention.

FIG. 17 is a perspective view of a preferred embodiment of a vaginal ring of the present invention.

FIG. 18 is a sectional view, partly in perspective and broken away, taken along lines 18-18 in FIG. 17.

FIGS. 19-20 are perspective views of alternative preferred embodiments of vaginal devices of the present invention.

FIG. 21 is a perspective view of a preferred embodiment of a vaginal ring of the present invention.

FIG. 22 is a perspective view of a preferred embodiment of a vaginal suppository of the present invention.

FIG. 23 is a perspective view of a preferred embodiment of a diaphragm of the present invention.

FIG. 24 is a sectional view taken along line 24-24 in FIG. 23 and broken away.

FIG. 25 is a perspective view of a preferred embodiment of an alternative diaphragm of the present invention.

FIG. 26 is a sectional view, broken away, taken along line 26-26 in FIG. 25.

FIGS. 27-28 are sectional views, broken away, analogous to FIG. 26 but showing alternative embodiments of diaphragms of the present invention.

### DETAILED DESCRIPTION OF THE PREFERRED EMBODIMENT

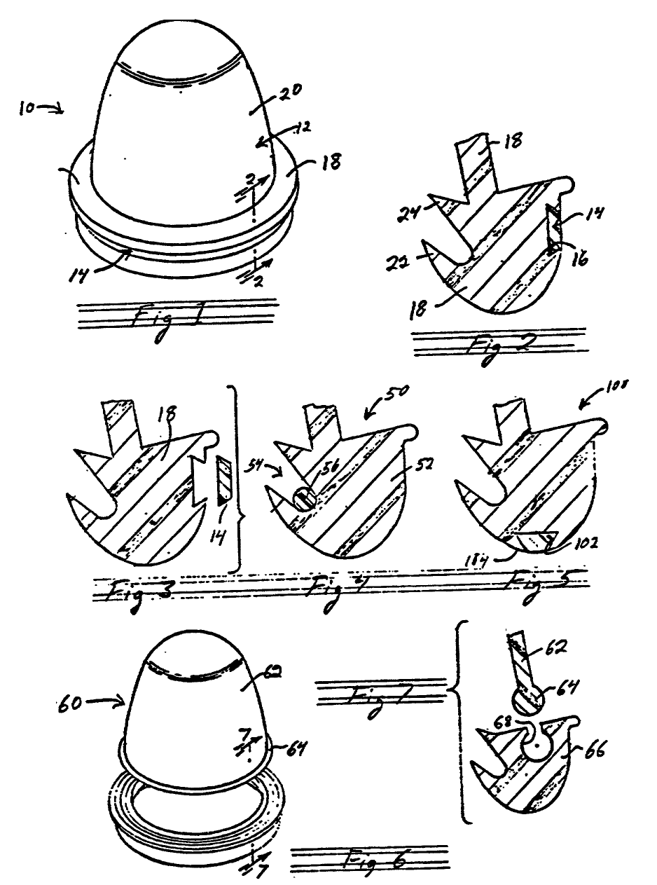

Now referring to FIG. 1, a preferred embodiment of a vaginal device of the present invention is shown in perspective view and indicated generally by the numeral 10. Vaginal device 10 is a cervical cap which, broadly speaking, has a dome 12 with an annular ring 14 retained thereon. As best shown in FIG. 2, annular ring 14 is snap-fit into, and retained by, groove 16 in annular rim 18 of dome 12.

Dome 12 is generally thimble-shaped and sized to fit over the cervix in a manner conventional for cervical caps. Annular rim 18 is integrally molded with thin, flexible non-resilient cap portion 20 of dome 12. Annular rim 18 has generally inwardly and upwardly, as viewed in FIG. 2, directed nibs 22 and 24 for gripping the cervix wall to retain vaginal device 10 in position over the cervix. Groove 16 extends around rim 18 on the radially outside face of annular rim 18. Groove 16 and annular ring 14 are shaped in cross-section and sized so that annular ring 14 in snappingly retained groove 16.

As is illustrated in the exploded view of FIG. 3, it is contemplated that annular ring 14 will be made as an element separate from rim 18 of dome 12 and then assembled with dome 12 to provide a complete vaginal device 10, a cervical cap.

It is contemplated that dome 12 will preferably be made of a non-resilient flexible material, such as a silicone based material, and that annular ring 14 will comprise a similar or the same material but be loaded, i.e., impregnated, with a desired biologically active material such as a medicament which will be released therefrom in an amount effective to achieve its purpose during use.

Types of silicone based materials suitable for use herein are known in the art and include high-consistency and low-consistency silicone-based elastomers prepared using a variety of well-known methods, e.g., platinum-cured systems, selected for compatibility with biological tissue and particular active ingredients being released by the device. An example of a biologically-active agent that could be released by the device is nonoxynol-9, commonly known as “N-9”, an anti-viral compound often used with contraceptive devices. The elastomer can be loaded with the active agent in a manner appreciated by those skilled in the art that incorporates the agent in an excipient matrix with the elastomer, providing sustained release of the agent from the matrix.

Further alternative embodiments of vaginal device 10 are illustrated the remaining FIGS. 4-28. The vaginal device 50 of FIG. 4 is of a construction analogous to vaginal device 10 of FIGS. 1-3 except that annular rim 52 has groove 54 carrying O-ring 56 which comprises a flexible material loaded or impregnated with medicament.

FIG. 5 illustrates another preferred embodiment of a vaginal device of this invention vaginal device 100 which is analogous to vaginal device 10 except that groove 102 extends around face 104 to retain annular ring 106 therein.

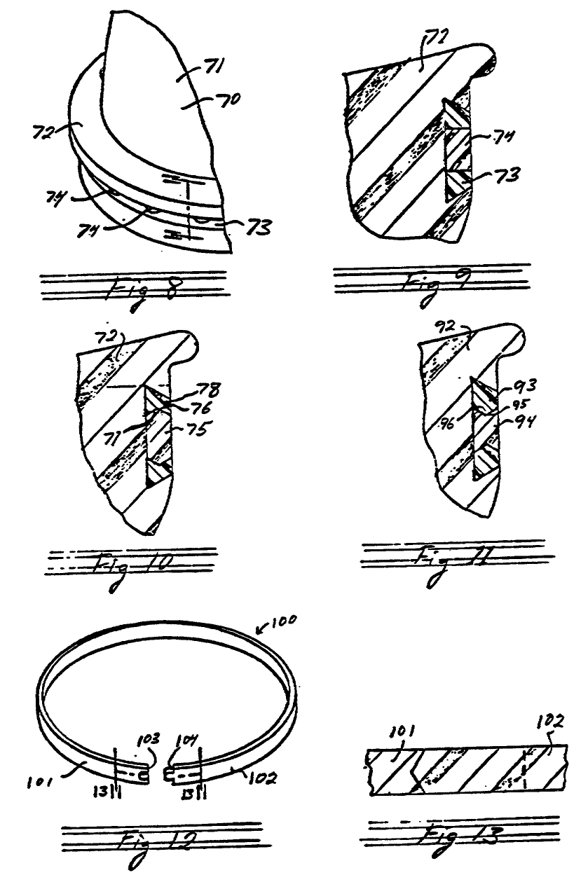

Now referring to FIGS. 6 and 7, another alternative embodiment of the present invention is shown and indicated generally by the numeral 60. Vaginal device 60 is a cervical cap having a dome portion 62 the open end of which terminates in an annular, bulbous edge 64. Bulbous edge 64 of dome portion 62 is adapted to snap-fit into rim 66. Thus, vaginal device 60 comprises a first part dome 62 and a second part rim 66. Rim 66 has an annular groove 68 adapted to receive and snappingly hold enlarged edge 64 of dome 62 to retain the two parts together.

FIGS. 8 and 9 illustrate another alternative embodiment of the present invention wherein a vaginal device 70 has a dome 71 and an annular rim 72 having an annular ring 73 snap-fit therein. Annular ring 73 in turn comprises a plurality of buttons 74 press fit therein. Thus a cervical device 70 comprises a plurality of parts including dome 71, annular ring 73 and a plurality of buttons 74. Annular ring 73 and each of buttons 74 can comprise the same of different biologically active materials.

FIGS. 10 and 11 are sectional views analogous to FIG. 9 but showing alternative arrangements for means for retaining buttons 74 in annular ring 73. Thus in the embodiment shown in FIG. 10 a button 75 has shoulders 76 which fit in corresponding recesses 77 in annular ring 78 to provide a snap fit therein.

In FIG. 11, annular ring 92 which is analogous to annular ring 72 of FIG. 8, carries annular ring 92 which is snap-fit therein. A plurality of buttons 94 are in turn snap-fit into annular ring 93. Thus, as is shown in FIG. 11, annular ring 93 has shoulders 95 which fit into corresponding recesses 96 in ring-like buttons 94 to provide a snap-fit for buttons 94 into ring 93.

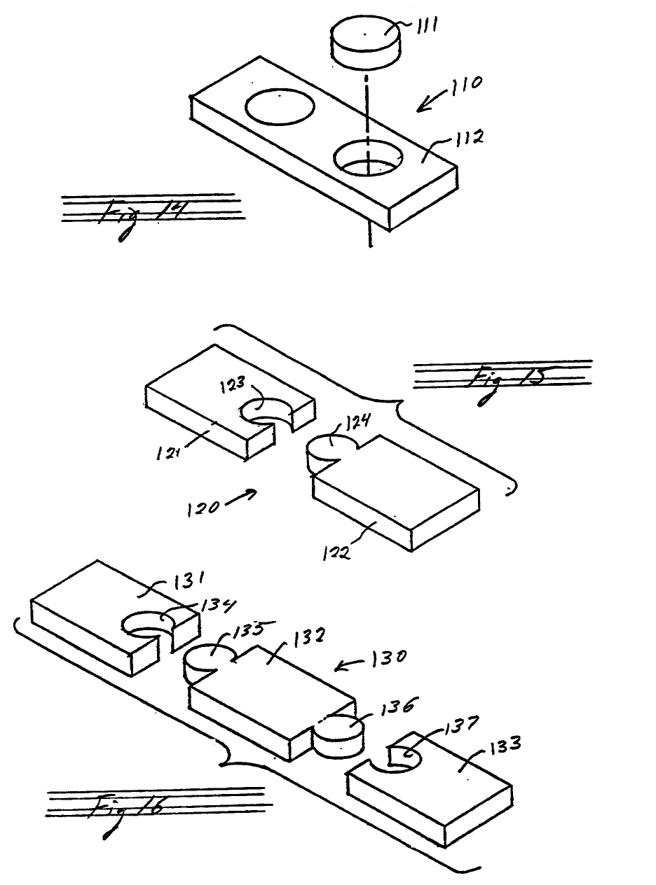

FIG. 12 shows a vaginal device 100 which is a vaginal ring comprising a first part 101 and a second part 102. End 103 of part 101 snap-fits into end 104 of part 102 as is illustrated in FIG. 13.

FIG. 14 shows a vaginal device 110 having a plurality of buttons 111 fit into strip 112.

FIG. 15 shows a vaginal device 120 comprising a first part 121 and a second part 122 each of which are in strip form. Part 121 has a recess 123 into which snappingly fits a projection 124 of part 122.

FIG. 16 shows a three part vaginal device 130 comprising a first strip 131, a second strip 132 and a third strip 133. First strip 131 has a recess 134 into which snappingly fits projection 135 of strip 132. Strip 132 also has a projection 136 at the end opposite thereof from projection 135. Projection 136 fits into a corresponding recess 137 and strip 133.

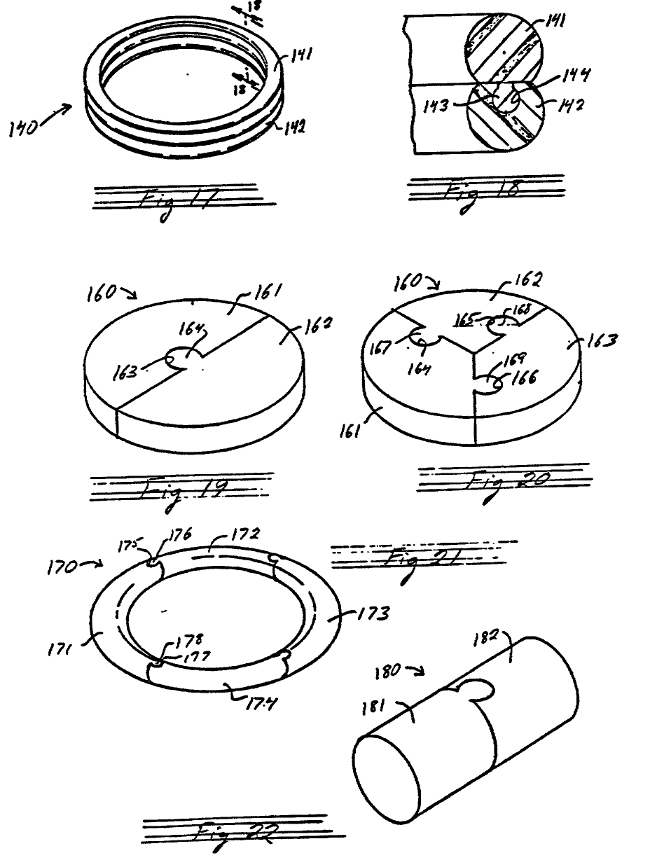

FIG. 17 shows a vaginal ring 140 comprising a first annular piece 141 and a second annular piece 142. As is best shown in FIG. 18, first annular piece 141 has a projection 143 which snappingly fits into a corresponding recess 144 in annular piece 142.

Now referring to FIGS. 19 and 20, two alternative embodiments of vaginal devices of the present invention are shown and indicated as vaginal device 150 and 160, respectively. Vaginal device 150, as shown in FIG. 19, comprises two disc halves 151 and 152. Vaginal disc half 151 has a recess 153 into which snappingly fits projection 154 of disc half 152. The vaginal device 160 as shown in FIG. 20 comprises three parts, vaginal disc third 161, vaginal disc third 162 and vaginal disc third 163. The vaginal disc thirds have recesses 164, 165, and 166 into which snappingly fit projections 167, 168 and 169.

FIG. 21 illustrates a vaginal device 170 which is a vaginal ring comprising four parts or segments 171, 172, 173 and 174. The opposite ends of each segment are provided with means for snappingly attaching to the adjacent opposite end of another segment. Thus segment 171 has one end having recess 175 therein which snappingly receives projection 176 of segment 172. The other end of segment 171 has projection 178 therein which snappingly fits into recess 177 of segment 174. Each of segments 172, 173 and 174 have corresponding means at each end to snappingly retrain adjacent ends of other segments.

FIG. 22 illustrates a vaginal device 180 comprised of two parts 181 and 182 and suitable for use as a vaginal suppository. Length 181 snappingly engages length 182.

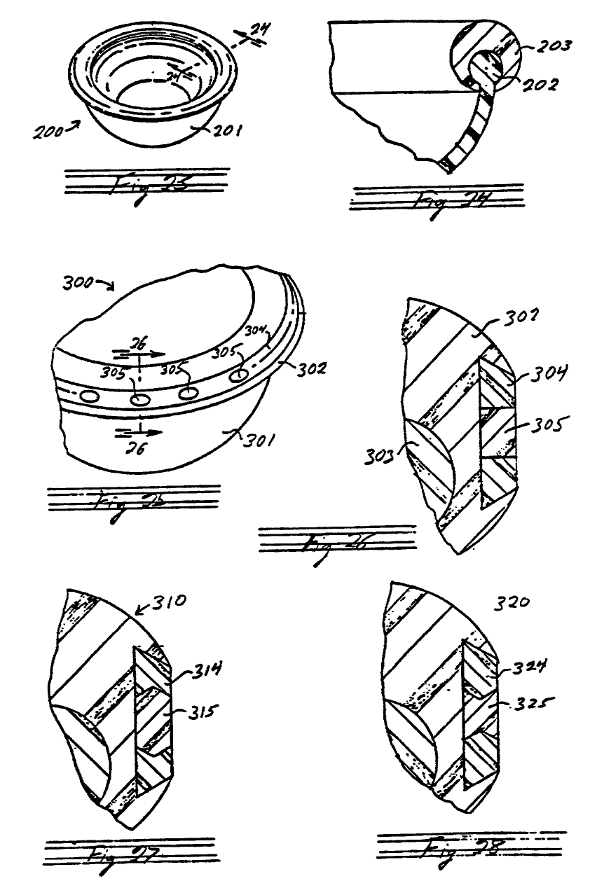

FIGS. 23-28 illustrate alternative embodiments of vaginal devices of the present invention which are diaphragms. The vaginal device 200 of FIG. 23 has a dome portion 201 with an enlarged rim 202 which is snappingly retained within an annular ring 203.

The vaginal device 300 shown in FIG. 25 is a diaphragm having a dome 301 with an annular rim 302 press fit over enlarged annular edge 303. Annular rim 302 in turn carries snappingly fit therein an annular ring 304 into which are press fit a plurality of buttons 305.

FIGS. 27 and 28 show alternative press fit arrangements. In FIG. 27 vaginal device 310 which is analogous to vaginal device 300 has an annular ring 314 which carries a plurality of buttons 315. In FIG. 28 vaginal device 320 has an annular ring 324 which carries a plurality of buttons 325.

From the above description of variations of the present invention it will be appreciated that the present invention is subject to substantial modification and variation by one skilled in the art within the scope of the present invention. Therefore, it is intended that the present invention be limited only by the scope of the appended claims.

What is claimed is:

1. A vaginal device having a first member and a second member, at least one of said members comprising a releasable biologically active material, said members being selectively attached to each other.

2. A vaginal device as in claim 1 wherein said first and second members each are comprised of a silicone based material.

3. A vaginal device as in claim 2 wherein said biologically active material is a medicament.

4. A vaginal device as in claim 2 wherein said biologically active material is a spermicide.

5. A vaginal device as in claim 3 wherein both of said first and second members comprise a releasable biologically active material.

6. A vaginal device as in claim 5 wherein said biologically active materials are different materials.

7. A vaginal device as in claim 1 wherein said device is a cervical cap and said first member is a form assuming non-resilient pliable dome with a flexible annular rim and said second member is retained in said rim.

8. A vaginal device as in claim 7 wherein said second member is an annular ring snap-fit into said annular rim.

9. A vaginal device as in claim 8 wherein said annular ring has a trapezoidal cross-sectional shape.

10. A vaginal device as in claim 7 wherein said annular ring has a circular cross sectional shape.

11. A vaginal device as in claim 8 wherein said annular ring has a rectangular cross-sectional shape.

12. A vaginal device as in claim 7 wherein said second member comprises a button press fit into said rim.

13. A vaginal device as in claim 1 wherein said device is a cervical cap, said first member is a form assuming pliable dome with a flexible annular rim and said second member is an annular ring retained over said rim.

14. A vaginal device as in claim 7 wherein said second member has a third member retained therein, said third member comprising a releasable, biologically active material.

15. A vaginal device as in claim 13 wherein said third member is a button press fit into said second member.

16. A vaginal device as in claim 1 wherein said device is a vaginal ring in the shape of an elongated belt with opposite first and second ends which snap-fit together to form a ring structure, said first end comprising the first member and said second end comprising the second member.

17. A vaginal device as in claim 16 wherein said vaginal ring comprises a third member press fit into an aperture in one of said first and second members.

18. A vaginal device as in claim 16 wherein said vaginal ring comprises a third elongated belt snap-fit between said first and second ends.

19. A vaginal device as in claim 1 wherein at least one of said first and second members is an elongated strip.

20. A vaginal device as in claim 19 wherein said first member is an elongated strip, said second member comprises a biologically active material and is a button press fit into said first member.
